(19) United States
(12) Patent Application Publication
(10) Pub. No.: US 2006/0058589 A1
(43) Pub. Date: Mar. 16, 2006

(54) Animal model to evaluate visceral pain perception

(76) Inventor: Maria Johanna Magdalena Aldina Nijsen, Beerse (BE)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 10/531,557

(22) PCT Filed: Oct. 13, 2003
(86) PCT No.: PCT/EP03/11329

§ 371 Date: Apr. 14, 2005

(30) Foreign Application Priority Data
Oct. 15, 2002 (WO) PCT/EP02/11772
May 7, 2003 (WO) PCT/EP03/04851

(51) Int. Cl. A61B 5/00

(52) U.S. Cl. 600/301

(57) ABSTRACT

Animal model to measure visceral pain of a balloon catheter and an implantable sensor module having transcutaneous telemetring ability. The implantable sensor module is set up to receive both visceromotor and pseudoaffective responses of the test animal. In particular, the balloon catheter is an implantable balloon catheter, preferably implanted in the duodenum of the test animal and the implantable sensor is set up to receive input signals form at least one bipolar electrode pair and at least one blood catheter. A method for producing said animal as well as kits comprising a balloon catheter and an implantable sensor module for use in a method for producing said animals are also described.

This invention relates to an animal model to measure visceral pain by means of a balloon catheter and an implantable sensor module having transcutaneous telemetring ability. The implantable sensor module according to the invention is set up to receive both visceromotor and pseudoaffective responses of the test animal. In particular, this invention provides a non-human animal model wherein balloon catheter is an implantable balloon catheter, preferably implanted in the duodenum of the test animal and the implantable sensor is set up to receive input signals form at least one bipolar electrode pair and at least one blood catheter. In particular this bipolar electrode is set up to receive visceromotor responses, especially electromyography of the abdominal muscle and the blood catheter set up to register mean arterial pressure and heart rate of the abdominal aorta.

It is thus a further object of the present invention to provide a method for producing said animal as well as kits comprising a balloon catheter and an implantable sensor module for use in a method for producing said animals.

### BACKGROUND OF THE INVENTION

The International Association for the Study of Pain has defined pain in the following way: “Pain is an unpleasant sensory and emotional experience associated with actual or potential tissue damage, or described in terms of such damage (Mertz 1979)”. The problem, however, is that pain cannot be measured directly in animals, but can only be estimated by examining their responses to nociceptive stimuli. Most models of nociception are based on behavioral responses to pain, ranging from the most elementary motor reflexes to far more integrated behaviors (escape, avoidance). A further problem with abdominal pain is that it is characterized by poor localization, abdominal cramps (visceromotor response) and autonomic (pseudoaffective) responses, including changes in respiration, heart rate (HR) and mean arterial pressure (MAP) that are difficult to score in a quantifiable and reproducible way.

The algesic writhing model has been used most commonly to study visceral pain in animals (Reichert, Daughters et al. 2001). In this model, an algesic solution is injected intraperitoneally into an awake animal and the number of writhes (stretches of the torso, hyperextension of the hind limbs with concave arching of the back and abdominal contractions) is scored. Due to ethical constraints, repeated assessments in a single animal cannot be performed, thereby compounding the difficulty of assessing tolerance development to analgesic agents. Furthermore, this model lacks escapability, specificity and is not related to human pathology. Mechanical induced stimuli of the viscera (distention of hollow viscera) reproduce a natural visceral stimulus, which mimics more closely visceral pain in humans, and is found aversive (avoidance/writhing behavior) in animals (Gebhart and Ness 1991; Ness, Randich et al. 1991; Ozaki, Bielefeldt et al. 2002). These visceral pain models produce quantifiable pseudoaffective reflexes, which include an increase in MAP and HR in the awake animal (Danzebrink and Gebhart 1990; Danzebrink and Gebhart 1991), although these are attenuated or even reversed by certain anesthetics (Ness and Gebhart 1988; Diop, Riviere et al. 1994; Ness 1999).

Colburn and colleagues (1989) studied the visceromotor response to volume-fixed duodenal distention in conscious, freely moving rats by scoring behavioural responses to pain such as shaking, exploring, grooming abdominal region, stretching or immobility and by scoring the occurrence of abdominal contractions. They demonstrated a graduated relationship between distending volume and the frequency of abdominal cramps. Furthermore, they showed that the visceromotor response to duodenal distention was inhibited by morphine in a dose-dependent manner.

In order to have a more quantifiable and reproducible model to study the visceromotor response to mechanical distention, several research groups are recording abdominal electromyography (EMG). Mostly, electrode wires were inserted into the abdominal or neck musculature and were exteriorized on the back of the animal from where it could be connected to an ink-writer or computer for EMG recording. These studies are done in the conscious restrained (Friedrich and Gebhart 2000; Ozaki, Bielefeldt, Sengupta, and Gebhart 2002; Bradesi, Eutamene et al. 2002) or lightly anaesthetized (Ness, Lewis-Sides et al. 2001) rat, to prevent damage of the exteriorized part of the balloon catheter and electrode leads due to biting and/or to minimalize background EMG noise induced by additional body movements (like exploration, grooming). However, in these studies both visceromotor and pseudoaffective responses to visceral pain are affected by the presence of anesthesia (Ness and Gebhart 1988; Ness 1999) and/or (restraint/handling) stress (Coutinho, Plotsky et al. 2002).

It would accordingly be, interesting to have an animal model of abdominal nociception to analyze analgesic properties of new pharmaceutical compounds, wherein the usefulness of such a visceral pain model is determined by the following criteria (Ness and Gebhart 1990; Gebhart and Sengupta 1996): (1) the stimulus should reproduce as much as possible a natural stimulus and must produce pain in humans, (2) the stimulus must induce aversive animal behavior (escape, withdrawal, avoidance), (3) the stimulus must evoke pseudoaffective responses consistent with those in humans in response to visceral pain, (4) responses to the stimulus must be modulated by antinociceptive manipulations, (5) the responses should be quantifiable and reproducible and (6) the model should be as non-invasive as possible and able to be used in anaesthetized animals.

All of the models described above only partially meet these requirements. It is thus an object of the present invention to provide a new animal model to study visceral pain especially characterized in that it can be used for both acute and chronic analysis of analgesic properties and that it allows simultaneous and continuous measurement of both the visceromotor (abdominal EMGs) and pseudoaffective response (MAP and HR) in a conscious, freely moving animal.

The present invention solves this problem by using a chronically implanted balloon catheter in the duodenum to deliver duodenal distention and a chronically implanted transmitter connected to a bipolar electrode pair and blood catheter for simultaneous and continuous telemetric measurements of the visceromotor (abdominal EMGs) and pseudoaffective response (MAP and HR) respectively.

### SUMMARY OF THE INVENTION

This invention relates to an animal model to measure visceral pain by means of a balloon catheter and an implantable sensor module having transcutaneous telemetring ability. The implantable sensor is capable to transmit data relevant to visceral pain to an apparatus outside the body capable of continuously monitoring the user's status and is capable to accept a plurality of input signals either simultaneously or sequentially, preferably from bipolar electrode pairs and blood catheters. As such, this model provides an adequate tool to measure visceromotor and pseudoaffective responses to visceral pain continuously and simultaneously in a non-human animal.

In a further embodiment this invention provides kits comprising a balloon catheter, an implantable telemetric sensor module, a bipolar electrode pair and a blood catheter, for use in a method to produce an animal model to measure visceral pain. It is also an object of the present invention to provide a balloon catheter for use in the aforementioned animal model.

It is thus a further object of the present invention to provide a method for producing an animal model to measure visceral pain comprising; implanting a balloon catheter in the duodenum of said animal; and implanting a telemetric sensor module in the abdominal cavity of said animal wherein said telemetric sensor module is set up to receive input signals from at least one bipolar electrode pair and at least one blood catheter.

### BRIEF DESCRIPTION OF THE DRAWING

FIG. 1. Drawing of the intra-gastroduodenal part of the silicone balloon catheter in uninflated and inflated condition.

FIG. 2. Changes in gross activity (counts/min) induced by staircase increases in distention volume (ml). During each distention period the behavioural observations are presented. bas=baseline and post=post distention period. Data are presented as means±SEM.

FIG. 3. Threshold volumes of distention (ml) to induce discomfort behaviour, pain behaviour, increase and decrease in baseline EMG signal. Data are presented as means±SEM.

FIG. 4. Individual tracing of a raw, filtered and rectified EMG waveform before, during and after staircase distention (0.1 to 0.6 ml).

FIG. 5. Changes in maximal amplitude of EMG (MAX) and area under curve (AUC) as percentage to baseline (=100%) induced by staircase increases in distention volume (ml). Data are presented as means±SEM.

FIG. 6. Changes in mean arterial pressure (MAP in mm Hg) and heart rate (HR in beats/min) induced by staircase increases in distention volume (ml). During each distention period the behavioural observations are presented. bas=baseline and post=post distention period. Data are presented as means±SEM.

FIG. 7. Correlation between mean arterial pressure (MAP in mm Hg) and MAX (mV) or AUC (mV×sec) during the staircase distention model.

FIG. 8. Changes in gross activity (counts/min) induced by phasic increases in distention volume (ml). During each distention period the behavioural observations are presented. bas=baseline and int=un-inflated interval. Data are presented as means±SEM.

FIG. 9. Individual tracing of a raw, filtered and rectified EMG waveform before, during and after phasic distention (0.1, 0.3 and 0.5 ml).

FIG. 10. Changes in maximal amplitude of EMG (MAX) and area under curve (AUC) as percentage to baseline (=100%) induced by phasic increases in distention volume (ml). Data are presented as means±SEM.

FIG. 11. Changes in mean arterial pressure (MAP in mm Hg) and heart rate (HR in beats/min) induced by phasic increases in distention volume (ml). During each distention period the behavioural observations are presented. bas=baseline and int=un-inflated interval. Data are presented as means±SEM.

FIG. 12. Correlation between mean arterial pressure (MAP in mm Hg) and MAX (mV) or AUC (mV×sec) during the phasic distention model.

FIG. 13. Changes in maximal amplitude of EMG (MAX) and area under curve (AUC) as percentage to baseline (=100%) induced by phasic increases in distention volume (ml) after pre-treatment with morphine (0.3, 1.5 and 3 mg/kg). Data are presented as means±SEM.

FIG. 14. Step-down latency (sec) of distended (0.6 ml) and non-distended rats exposed to a small platform in an open field for 9 trails. Data are presented as means±SEM.

### DETAILED DESCRIPTION

This invention relates to an animal model to measure visceral pain by means of a balloon catheter and an implantable sensor module having transcutaneous telemetring ability. The implantable sensor is capable to transmit data relevant to visceral pain to an apparatus outside the body capable of continuously monitoring the user's status and is capable to accept a plurality of input signals either simultaneously or sequentially, preferably from bipolar electrode pairs and blood catheters. As such, this model provides an adequate tool to measure visceromotor and pseudoaffective responses to visceral pain continuously and simultaneously in a non-human animal. In particular it relates to a non-human animal model to measure visceral pain comprising a balloon catheter and an implantable sensor module having transcutaneous telemetring ability. Preferably, a non-human animal wherein both the balloon catheter and sensor module are chronically implanted in said animal. Preferably, the balloon catheter is implanted in the duodenum and the sensor module is connected to a bipolar electrode pair and a blood catheter.

The balloon catheter used in the present model is an implantable balloon catheter characterized in that the balloon catheter consists of biocompatible material such as non-immunogenic polymeric material of poly-para-xylylene, polyethylene, natural or synthetic rubber, silicone or other aromatic based moiety having a membrane portion with a porosity effective to block passage of immunogenic agents. The balloon catheter of. the present invention consists of biocompatible tubing (1) closed at one end with elastic material (2). To improve the introduction of the balloon catheter into the animal according to the method of the present invention, the elastic material is attached to the biocompatible tubing at a position (3) proximal from the tube end (4). In a further improvement to enhance the radial expansion of the elastic material, the elastic material is also attached at the end of the biocompatible tubing (5) and said tubing end is rigidly sealed (6). To allow balloon inflation in this last configuration, the tubing end distal from attachment point (3) is foreseen from a number of holes (7). A further characteristic of the balloon catheter according to the invention is that it comprises fixation means to prevent movement of the catheter due to the peristaltic movement of the intestinal tract. Said fixation means are positioned proximal from the tube end to allow fixation of the tubing to the stomach wall of the animal. In a preferred embodiment the fixation means consist of two nodes (8).

The diameter and length of the tubing are determined by the test animal used. As in the method according to the invention the implantable balloon catheter is introduced in the duodenum of the test animal, the length of the tubing should permit to reach from the duodenum of said animal, via the stomach wall, to the skull where it is accessibly immobilized. For example in rat the length of the tubing would be between 15 and 50 cm, preferably between 20 and 30 cm with an outside diameter from 2-2.5 mm.

It is thus a further object of the present invention to provide balloon catheter consisting of biocompatible tubing (1) closed at one end with elastic material (2), characterized in that the elastic material is attached to the biocompatible tubing at a position (3) proximal from the tube end (4). In a further embodiment the elastic material is also attached at the end of the biocompatible tubing (5) and said tubing end is rigidly sealed (6), further comprising a number of holes (7) distal from attachment point (3). In a particular embodiment the balloon catheter further comprises fixation means that are positioned proximal from the tube end to allow fixation of the tubing to the stomach wall of the animal. In a preferred embodiment the fixation means consist of two nodes (8).

The implantable sensor modules with transcutaneous telemetring ability that can be used in the animal model according to the invention are known in the art. For example, there are pacemakers available which, when implanted and connected to the heart, can monitor electrocardial activity through electrodes attached to the pacemakers. The electrodes function as electropotential sensors, and the pacemaker include interface circuits which buffer the sensor signals, formats them and transmits then the formatted signals by way of a bidirectional radiofrequence (RF) communication link to an external communication module. The telemetered signals are monitored and processed through the external module.

Further it is known in the art to provide for enablement of two or more functions with implanted telemetric devices allowing the sequential or simultaneous measurement of up to eight individual parameters. In the animal model according to the present invention the implantable sensor module is capable of accepting a plurality of input signals either sequentially or simultaneously. In a preferred embodiment the sensor module comprises at least two input ports and is connected to at least one bipolar electrode pair and at least one blood catheter. In a more preferred embodiment the sensor module is a radiotelemetric device connected to a bipolar electrode pair for electromyography measurement of the abdominal muscle and connected to a blood catheter set up to register mean arterial pressure and heart rate of the abdominal aorta through the femoral artery. It will be readibly appreciated by the skilled person that the implantable sensor module as used herein requires an external module to detect and demodulate the output signal of the sensor module into an output signal suitable for driving an output graphics device. For example a recorder for recording the variations in amplitude of a current over time.

It is thus an object of the present invention to provide a system for measuring visceral pain comprising; a balloon catheter, means for introducing a measured volume of inflation medium through the proximal end of the balloon catheter, an implantable sensor having transcutaneous telemetring ability, and an external module capable to monitor and process the telemetered signals. A system wherein the balloon catheter is implanted in the duodenum of a test animal and wherein the implantable sensor module is set up to receive both visceromotor (EMG) and pseudoaffective (MAP, HR) responses of the test animal. In a particular embodiment the system comprises a radiotelemetric device connected to a bipolar electrode pair for electromyography measurement of the abdominal muscle and connected to a blood catheter set up to register mean arterial pressure and heart rate of the abdominal aorta through the femoral artery. The balloon cathether used in the aforementioned system typically consists of of biocompatible tubing (1) closed at one end with elastic material (2), characterized in that the elastic material is attached to the biocompatible tubing at a position (3) proximal from the tube end (4). In a further embodiment the elastic material is also attached at the end of the biocompatible tubing (5) and said tubing end is rigidly sealed (6), further comprising a number of holes (7) distal from attachment point (3). In a particular embodiment the balloon catheter further comprises fixation means that are positioned proximal from the tube end to allow fixation of the tubing to the stomach wall of the animal. In a preferred embodiment the fixation means consist of two nodes (8). The means for introducing a measured volume of inflation medium typically comprises a syringe including a fluid-infusion pump which is driven under the control of a servo system or a stepper motor, wherein the inflation medium is typically a sterile, relatively bubble-free and substantially incompressible fluid, such as a normale saline solution. In the present invention the volumes of inflation medium will be determined by the test animal used. For example in rat the volume of inflation medium is in the range of 0.05-1.00 ml, preferably in the range of 0.1-0.6 ml.

In a further embodiment this invention provides kits comprising a balloon catheter, an implantable telemetric sensor module, a bipolar electrode pair and a blood catheter, for use in a method to produce an animal model to measure visceral pain. Optionally, further comprising an external module capable to monitor and process the telemetered signals and/or means for introducing a measured volume of inflation medium. In a particular embodiment the kit comprises a balloon catheter according to the invention and the implantable telemetric sensor module is capable of accepting a plurality of input signals either sequentially or simultaneously and has transcutaneous telemetring ability. In a preferred embodiment the sensor module comprises at least two input ports.

It is thus a further object of the present invention to provide a method for producing an animal model to measure visceral pain comprising; implanting a balloon catheter in the duodenum of said animal; and implanting a telemetric sensor module in the abdominal cavity of said animal wherein said telemetric sensor module is set up to receive input signals form at least one bipolar electrode pair and at least one blood catheter. A process for producing an animal model according to the invention wherein the balloon catheter is fixated to the stomach wall of the test animal using the fixation means (8) of the implantable balloon catheter. A process for producing an animal model according to the invention wherein the biocompatible tubing (1) of the implantable balloon catheter is guided to the skull of the test animal, preferably subcutaneously, where it is accessibly immobilized. In a preferred embodiment the tubing end is fixed to a syringe connecter, preferably a connecter with a 90° loop which are commercially available. The syringe connecter is fixated to the skull of the test animal, for example using dental cement. A process for producing an animal model according to the invention wherein the electrodes of the bipolar electrode pair is sutured into the abdominal muscle for EMG measurements. A process for producing an animal model according to the invention wherein the blood catheter is tunneled into the abdominal aorta, preferably through the femoral artery.

This invention will be better understood by reference to the Experimental Details that follow, but those skilled in the art will readily appreciate that these are only illustrative of the invention as described more fully in the claims that follow thereafter. Additionally, throughout this application, various publications are cited. The disclosure of these publications is hereby incorporated by reference into this application to describe more fully the state of the art to which this invention pertains.

### EXPERIMNTAL

Material and Methods

Animals

Naive male albino Wistar rats (WU; Harlan, The Netherlands) weighing 280-300 g at the beginning of the experiments, were used. Rats were housed individually in a Macrolon individual ventilated cage (25×40×22 cm) containing a layer of wood shavings under conditions of constant ambient temperature (21±1° C.), constant humidity (60±15%) and light/dark rhythm (with lights on from 7 a.m. to 7 p.m.). After surgery, the animals were housed individually under presurgical conditions. Food (complete laboratory chow) and water were accessible ad libitum throughout the experiment.

Surgery

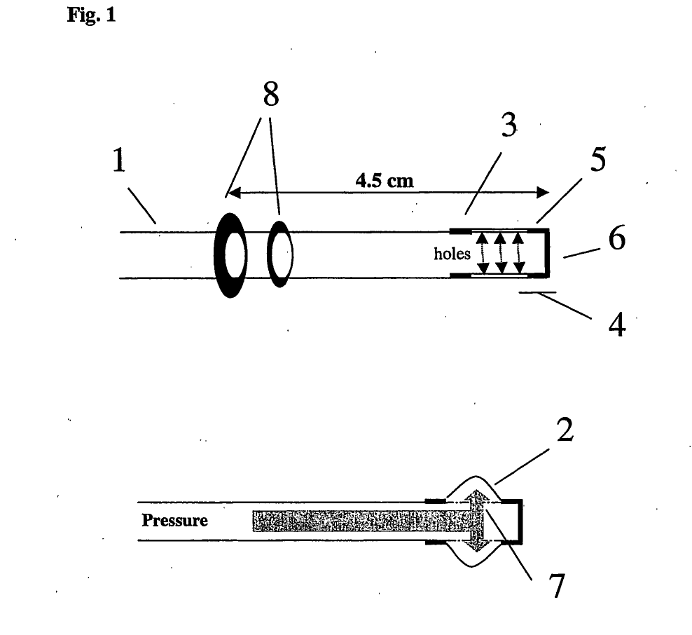

Rats were equipped with a balloon catheter in the duodenum to induce duodenal distention and a telemetric transmitter to study abdominal electromyography (EMGs), mean arterial pressure (MAP), heart rate (HR) and body activity. Operations were performed under fentanyl/fluanisone anesthesia (Hypnorm®, Janssen Pharmaceutica, Beerse, Belgium; 0.1 m/100 g body weight (BW), intramuscularly) and Midazolam hydrochloride (Dormicum®, Hoffman-LaRoche, Mijdrecht, The Netherlands; 0.05 ml, intraperitoneally (ip)) as a muscle relaxant. Before the muscle relaxant was injected, the analgesic effect of fentanyl anesthesia was tested in the rat by checking for the absence of its pedal and cornea reflexes. Total absence of the pain response normally appeared after 10 min and then the muscle relaxant was injected. Surgery was performed in a sterile laminar flow cabinet to minimize risk of infection. A small longitudinal incision was made on the linea alba at the anterior of the abdomen. A self-made silicone balloon catheter (i.d. 1.02 mm/o.d. 2 mm) was chronically implanted into the duodenum according to the procedure described by Colburn et al. (1989), with some modifications (see FIG. 1). A small incision (3 mm) was made in the stomach wall for insertion of the catheter, which in turn was guided into the duodenum until the small node on the balloon catheter has just crossed the stomach wall. Then the stomach wall was sutured tightly between the two nodes to fixate the catheter. The other end of the balloon catheter was tunneled subcutaneously to the skull, where it was fixed to a connector (Bilaney, Dusseldorf, Germany) with a 90° loop. There it was fixated to the skull with dental cement (Dentimex B V, Zeist, The etherlands). A telemetry transmitter (TL11M2-C50-PXT, Data Sciences International, St. Paul, USA), consisting of a bipolar electrode pair and blood catheter, was chronically implanted into the abdominal cavity of the rat. The non-insulated tips (helix of stainless steel wire; Ø 0.45 mm, 8 mm) of the electrodes were sutured in parallel (5 mm inter-electrode space) into the abdominal muscle for EMG measurements, whereas the blood catheter was tunneled through the femoral artery to the abdominal aorta to register MAP and HR. Postoperatively the animals received 0.1 mg/kg of the long-acting opiate analgesic Buprenorfine hydrochloride (Temgesic®, Reckitt & Colman; Kingston-upon-Hull, UK; 0.1 ml, subcutaneously).

Experimental Design

Rats (n=14) were surgically equipped with a duodenal balloon catheter and telemetric transmitter and were allowed to recover from surgery for 12 days. During the recovery period the animals were handled every day for weighing and habituation purposes. Rats were accustomed to experimental procedures (twice before the experiment). During the experiment, EMG, body activity, MAP and HR were simultaneously and continuously recorded before (baseline), during and after (post) duodenal distention. Baseline values were recorded for 30 sec. Subsequently, rats received a volume-fixed duodenal distention protocol of 300 (staircase n=7) or 690 (phasic n=7) sec (see below). Afterwards, post recordings were performed for another 120 (staircase) or 300 (phasic) sec.

To validate our model, an additional experiment was performed with a new group of rats. Thirty min prior to phasic duodenal distention, rats were ip injected with morphine (0.3 (n=8), 1.5 (n=7) or 3 (n=8) mg/kg body weight) or saline (n=8).

In a final experiment to validate our model, hereinafter referred to as the passive avoidance test, 12 rats were surgically equipped with a duodenal balloon catheter and were allowed to recover from surgery for 12 days. These animals were handled every day for weighing and habituation purposes. In the passive avoidance test, the step down latency was scored. All experiments were performed in the home cage during the light phase of the circadian cycle between 8 a.m. and 12 p.m. After the experiment, all rats were killed by an overdose (0.5 ml) of pentobarbital (Nembutal®, Sanofi, Belgium; 60 mg/ml, ip), dissected and macroscopically inspected for infections. In none of the animals any signs of infection were found.

Duodenal Distention

Forty-five min prior to baseline recordings, the skull connector of the balloon catheter was attached to a fluid-filled long-line (1 m; polyethylene tubing, Becton Dickinson, UK) with a syringe, so that variable volume-fixed distentions could be delivered from outside the home cage without restraining the rat. During this habituation period, rats remained to sleep, so that stress-free baseline recordings were started. Two protocols were used: 1) staircase distention: the balloon was inflated with increasing volumes of 0.1 ml (each 30 sec), starting from 0.1 to 0.6 ml; 2) phasic distention: inflation of 0.1, 0.3 and 0.5 ml for 30 sec with resting un-inflated intervals of 5 min.

Behavioral Measurements

During the experiment, the behavioral response to duodenal distention in the home cage was scored by the experimentator. Following behavioral scores were used: sleep, wake up, alert, shake, explore, groom abdominal region, abdominal contractions, stretching behavior (stretches of the torso and hyperextension of the hind limbs with concave arching of the back) and immobility. In the staircase model, the threshold (distention volume (ml)) inducing discomfort (shake, explore) and/or pain (groom abdominal region, stretching and immobility) behavior was determined.

Passive Avoidance Test

To validate whether duodenal distention is perceived as aversive, rats (n=12) were exposed to a passive avoidance test. On day 1, the rat was palced on a small platform, which was located in a large open field (Ø 80 cm). When the rat stepped down the platform, it was removed from the open field. For habituation purposes, this procedure was repeated for each rat in 9 learning trails with an interval of 10 min. When the rat did not step down the platform, it was removed after 150 sec. The next day, the same rats were submitted to the same procedure (no distention, control group; n=6) or received a duodenal distention of 0.6 ml for 5 sec immediately after stepping down the platform (n=6). This procedure was repeated in each rat for 9 learning trails with an interval of 10 min. An increase in step down latency can be used as an indication for increased pain perception.

Telemetric Measurements

Body activity was telemetrically measured by detecting changes in signal strength of the transmitter that occurred as the animal moved about its cage. EMGs, body activity, MAP and HR were registered by the data acquisition program ART 2.2 (Data Sciences International, St. Paul, USA). Raw EMG activity was continuously collected as a waveform (at a frequency of 1000 Hz), was low cut filtered at 50 Hz to eliminate movement interference and fully rectified by Spike2, version 4.11 (Cambridge Electronic Design, Cambridge, UK). From the rectified EMG, area under the curve (AUC; mV×sec) and maximal value (EMGₘₐₓ; mV) were analyzed. In the staircase model, the threshold (distention volume (ml)) inducing an increase or decrease in baseline EMG amplitude was determined.

Statistics

Distention thresholds (ml), EMG data (% to baseline), gross activity (counts/min), MAP (mmHg) and HR (beats/min) are presented as means±SEM. Cardiovascular data of each single rat are averaged to 1 point per baseline and distention period, before statistical analysis is performed by one-way Analysis of Variance (ANOVA) and post-hoc Student's t-test. For the morphine experiment, a two-factor multiple ANOVA (MANOVA) with repeated measured was used. P values of <0.05 were considered significant.

In the phasic distention protocol, one rat was excluded for statistical analysis on cardiovascular data due to bad signaling.

Results and Discussion

Staircase Model

Behavioral and Visceromotor Response

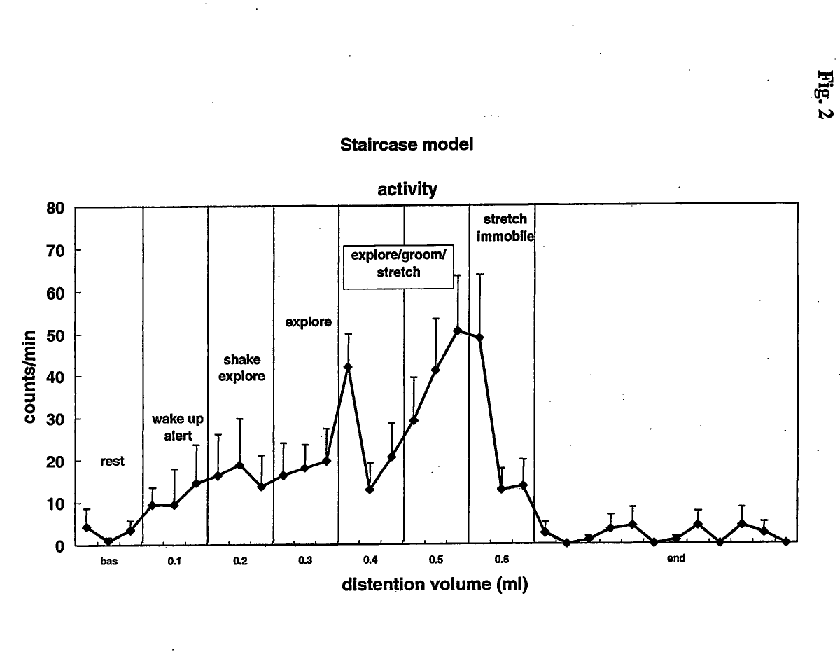

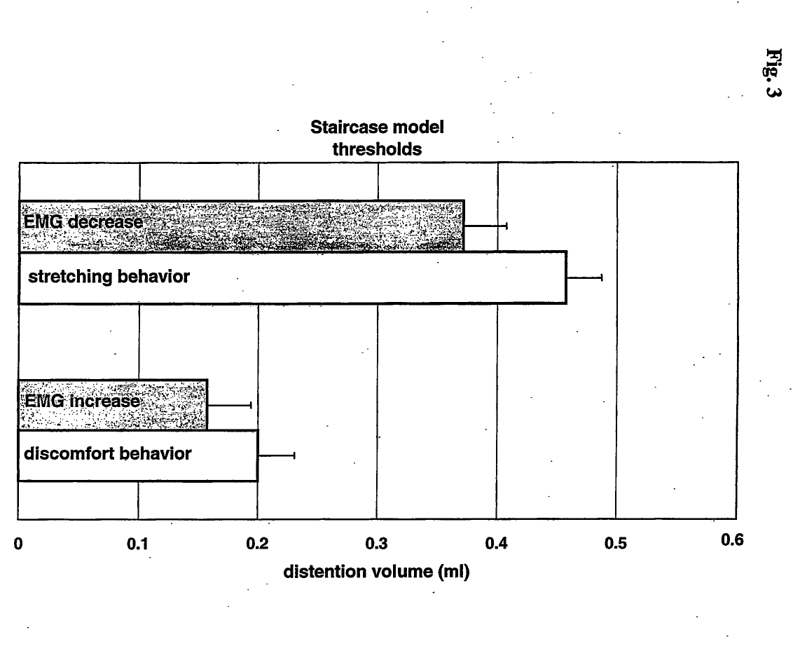

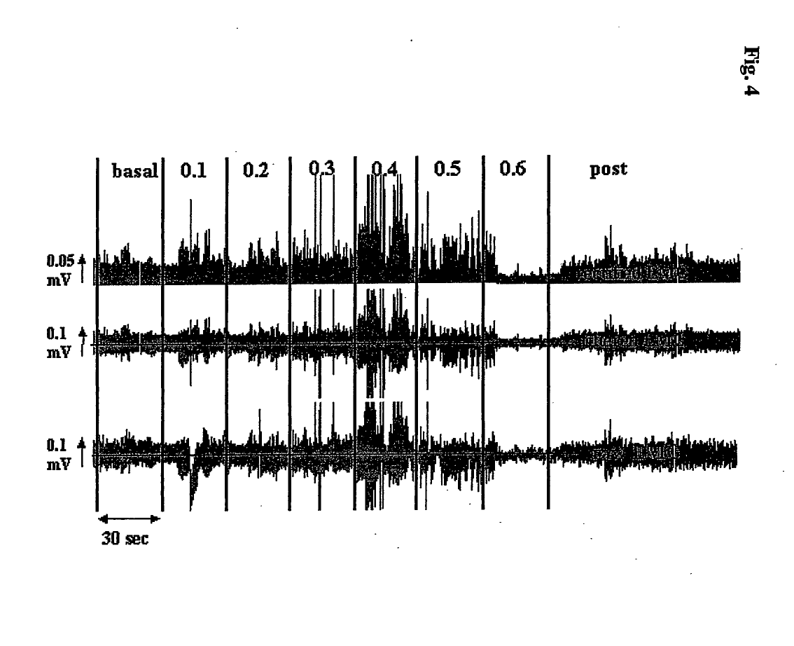

Staircase distention of the duodenum produced “discomfort” behavior (shaking, exploring) starting at a volume of 0.2 ml. At higher volumes (0.4 to 0.6 ml) rats showed “pain” behavior (grooming abdominal region, stretching, immobility) additionally (FIGS. 2 and 3). Abdominal contractions are shown before the occurrence of pain-related behavior. FIG. 4 shows an example of an individual tracing of the raw, filtered an rectified EMG signal before (basal), during (0.1 to 0.6 ml) and after (post) staircase distention. “Active” behavior (shaking, exploring, grooming, abdominal cramps) was accompanied by a significant increase in EMG amplitude, whereas stretching behavior during higher distention volumes (0.4 to 0.6 ml) was reflected in a decrease of the baseline EMG signal (FIGS. 3 and 4).

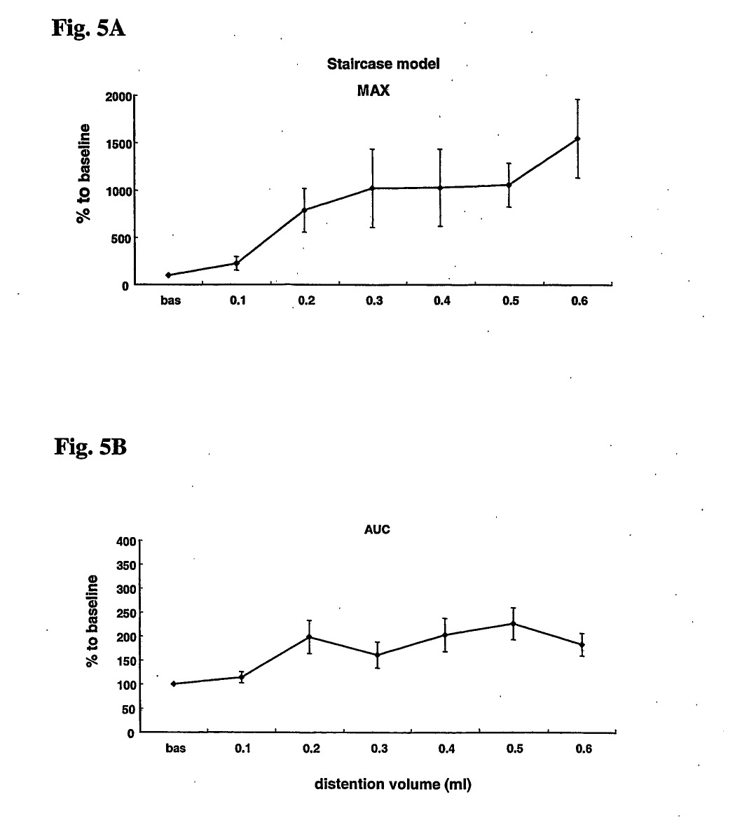

Staircase distention produced a volume-dependent increase in EMGₘₐₓ and AUC (FIG. 5). One-way ANOVA on EMGₘₐₓ (F(6, 48)=2.9, p<0.05) and AUC (F(6, 48)=3.1, p<0.05) showed significance and post-hoc analysis revealed a significant increase in AUC and EMGₘₐₓ as compared to baseline at 0.2 (p<0.05), 0.3 (p<0.05), 0.4 (p<0.05), 0.5 (p<0.005) and 0.6 (p<0.005) ml distention volume.

Cardiovascular Response

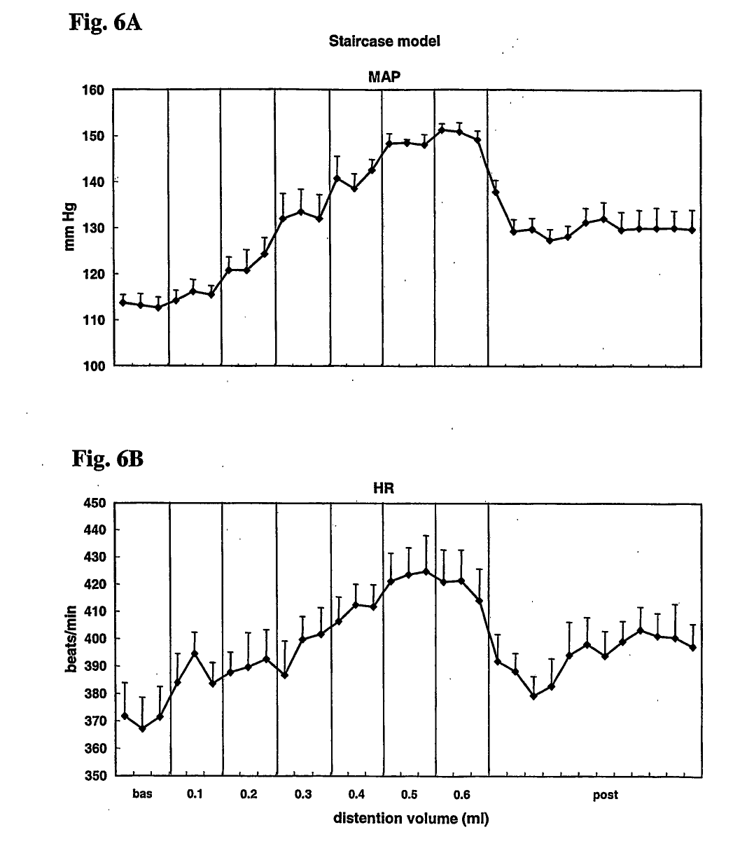

Staircase distention of the duodenum produced a volume-dependent increase in MAP (FIG. 6A). One-way ANOVA on MAP showed significance (F(6, 48)=22.6, p<0.0001) and post-hoc analysis revealed a significant increase in MAP as compared to baseline at 0.3 (p<0.005), 0.4 (p<0.0001), 0.5 (p<0.0001) and 0.6 (p<0.0001) ml distention volume.

Staircase distention of the duodenum produced a volume-dependent increase in HR (FIG. 6B). One-way ANOVA on HR showed significance (F(6, 48)=3.9, p<0.005) and post-hoc analysis revealed a significant increase in HR as compared to baseline at 0.3 (p<0.05), 0.4 (p<0.01), 0.5 (p<0.005) and 0.6 (p<0.01) ml distention volume.

Relationship Visceromotor—Pseudoaffective Response

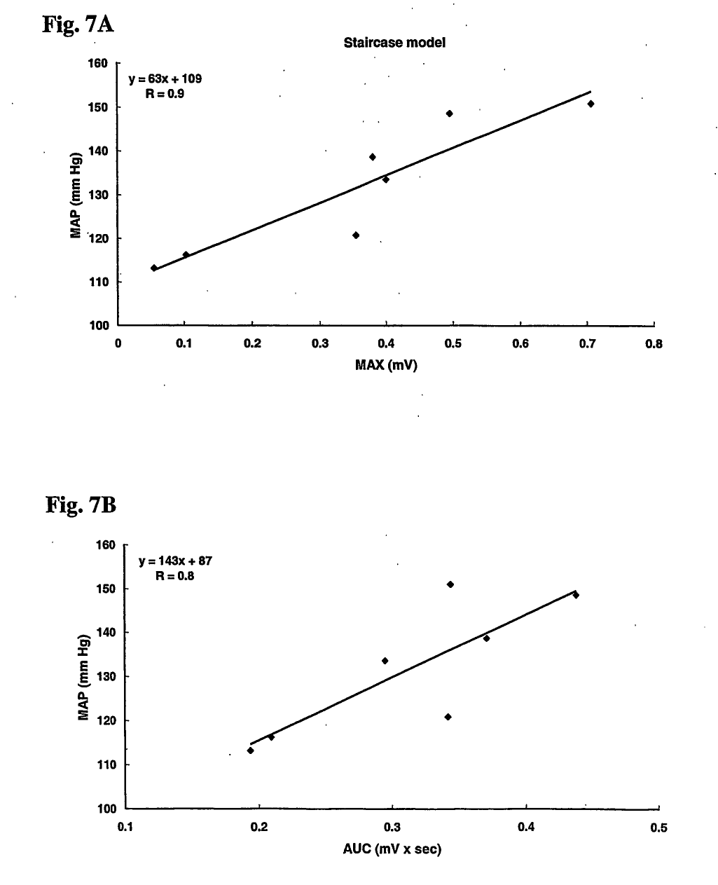

FIG. 7 shows the positive correlation between mean MAP and EMGₘₐₓ (MAP=63 EMGₘₐₓ+109, r=0.9) or AUC (MAP=143 AUC+87, r=0.8) during staircase distention. Mean MAP also showed a positive correlation with HR(HR=1.2 MAP+242, r=0.9) and mean HR showed a positive correlation with EMGₘₐₓ (HR=75 EMGₘₐₓ+375, r=0.8) and AUC (HR=185 AUC+343, r=0.8); data not shown.

Phasic Model

Behavioral and Visceromotor Response

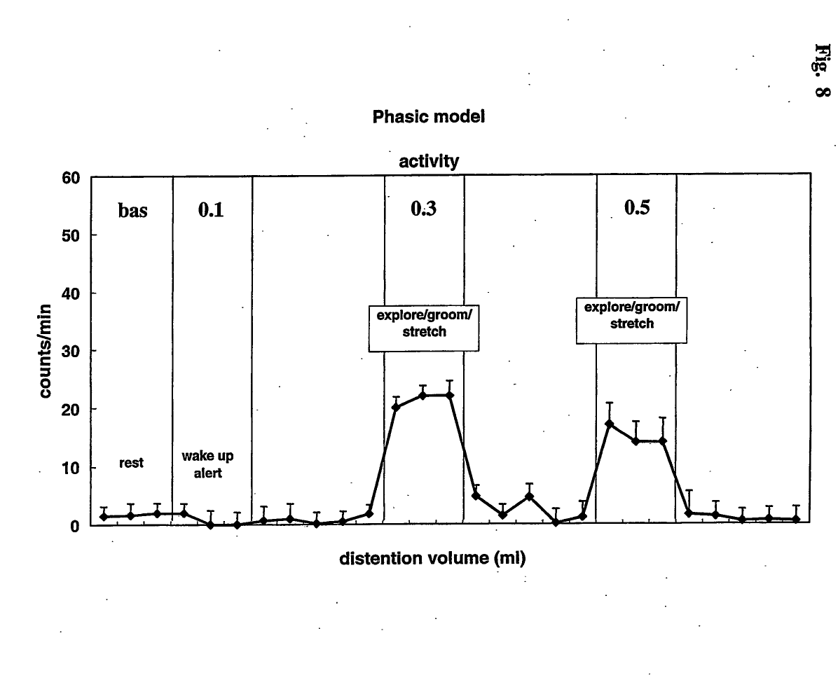

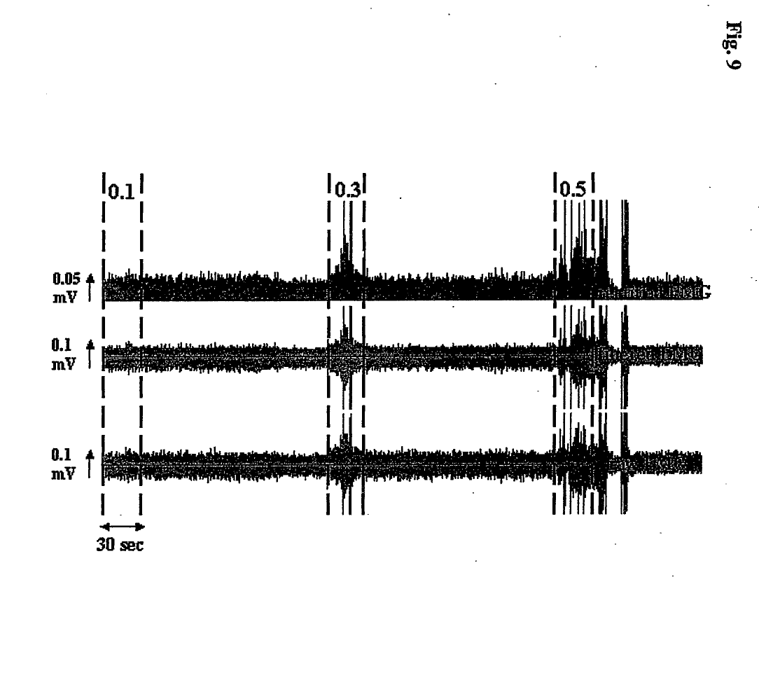

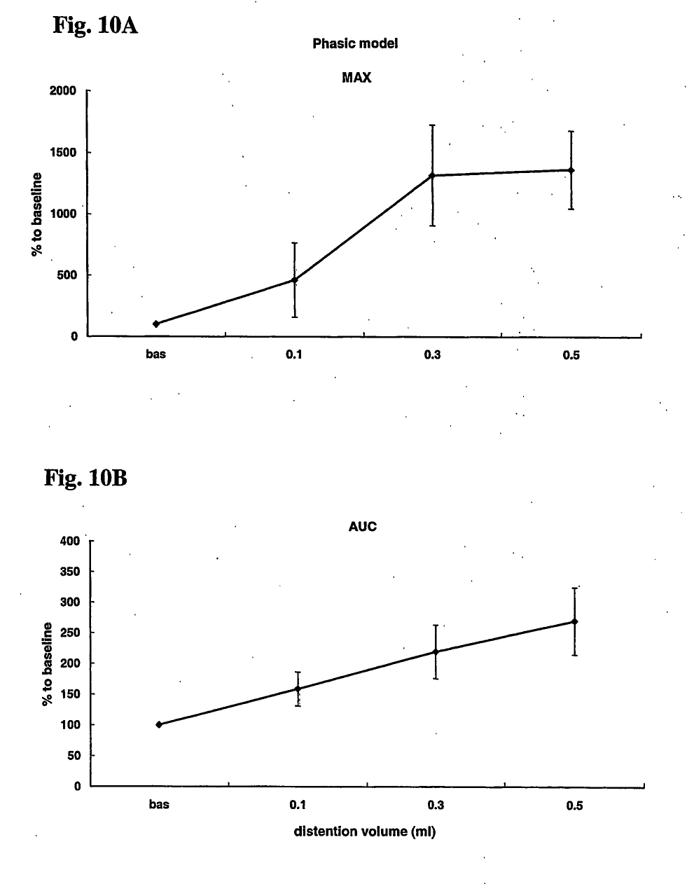

Phasic distention of the duodenum produced “discomfort” and “pain” behavior at a volume of 0.3 and 0.5 ml (FIG. 8). Stretching behavior was less reflected in a reduction of the basal EMG amplitude (see FIG. 9) as shown in the staircase protocol. Phasic distention produced a volume-dependent increase in EMGₘₐₓ and AUC (FIG. 10). One-way ANOVA on EMGₘₐₓ (F(3, 27)=4.4, p<0.05) and AUC (F(3, 27)=3.8, p<0.05) showed significance and post-hoc analysis revealed a significant increase in AUC and EMGₘₐₓ as compared to baseline at 0.3 (p<0.05) and 0.5 (p<0.01) ml distention volume.

Cardiovascular Response

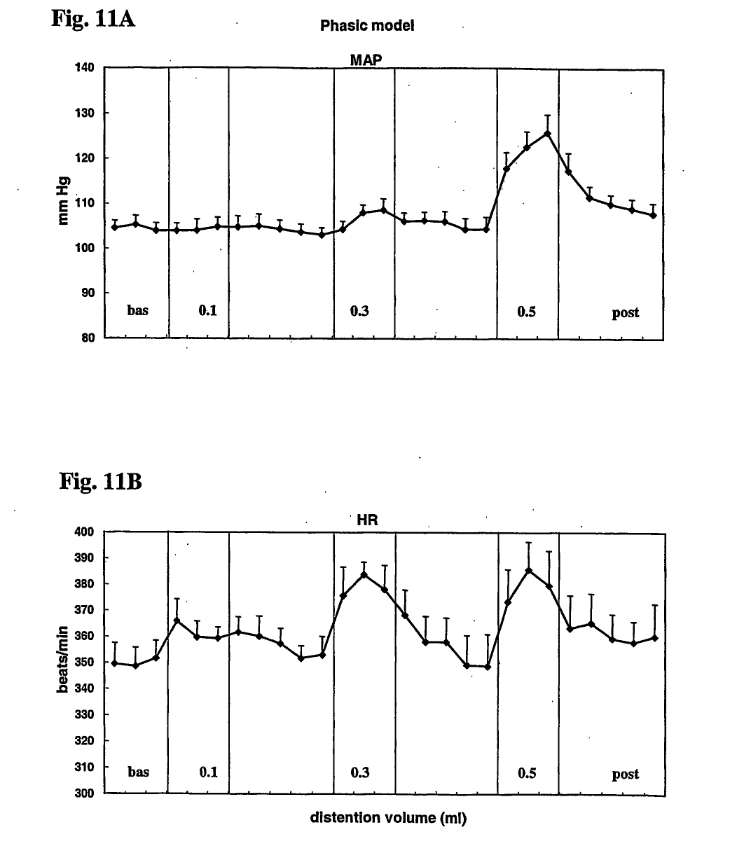

Phasic distention of the duodenum produced a volume-dependent increase in MAP (FIG. 11A). One-way ANOVA on MAP showed significance (F(3, 23)=11.8, p<0.0001) and post-hoc analysis revealed a significant increase in MAP as compared to baseline at 0.5 (p<0.005) ml distention volume.

Phasic distention produced a volume-dependent increase in HR (FIG. 11B). One-way ANOVA on HR showed significance (F(3, 23)=5.8, p<0.005) and post-hoc analysis revealed a significant increase in HR as compared to baseline at 0.3 (p<0.005) and 0.5 (p<0.05) ml distention volume.

Relationship Visceromotor—Pseudoaffective Response

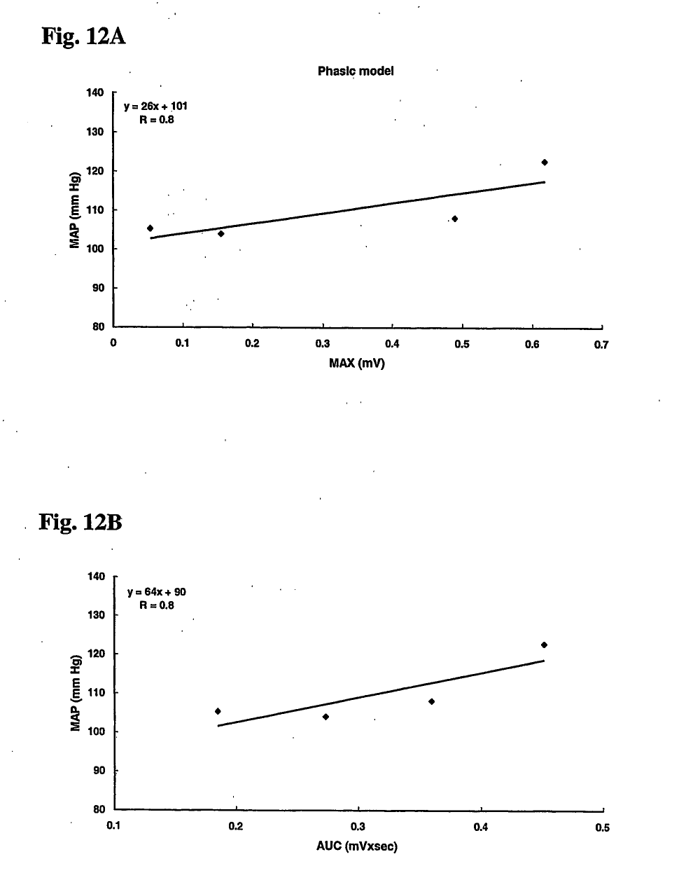

FIG. 12 shows the positive correlation between mean MAP and EMGₘₐₓ (MAP=26 EMGₘₐₓ+101, r=0.8) or AUC (MAP=64 AUC+90, r=0.8) during phasic distention. Mean MAP also showed a positive correlation with HR (HR=1.5 MAP+204, r=0.7) and mean HR showed a positive correlation with EMGₘₐₓ (HR=67 EMGₘₐₓ+347, r=0.98) and AUC (HR=152 AUC+321, r=0.95); data not shown.

Morphine Experiment

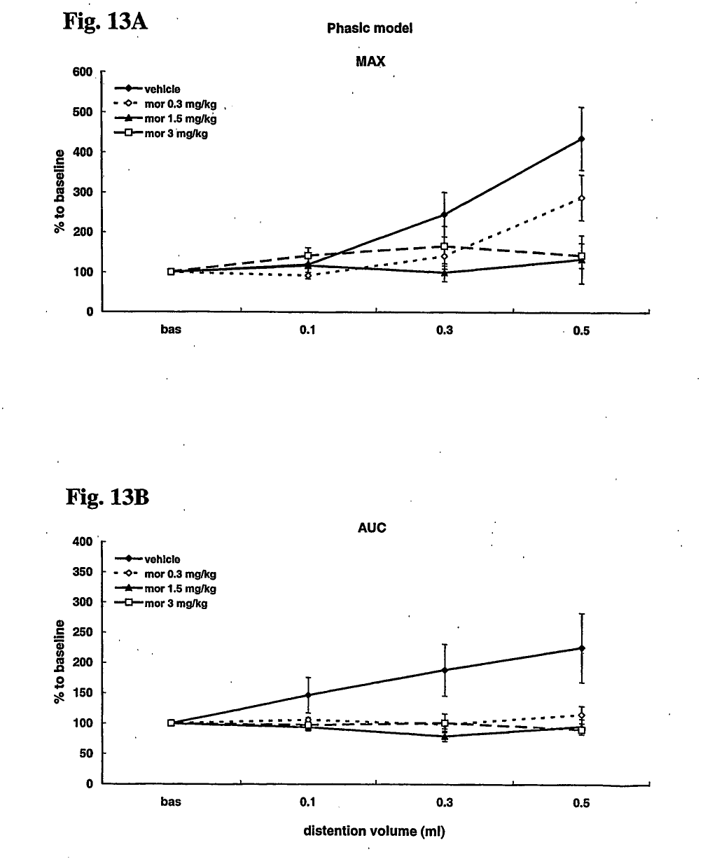

Pre-treatment with morphine inhibited distention-induced pain behavior (stretching behavior, grooming abdominal region, immobility; p<0.0001, data not shown). Morphine treatment dose-dependently reduced the distention-induced increase in EMGₘₐₓ and inhibited the distention-induced increase in AUC (FIG. 13). MANOVA revealed a significant treatment effect on EMGₘₐₓ (F(3,27)=4.8, p<0.01) and AUC (F(3,27)=4.3, p<0.05), significant distention effect on EMGₘₐₓ F(2,26)=14.9, p<0.0001) and AUC (F(2,26)=10.8, p<0.0005) and significant distention×treatment interaction on EMGₘₐₓ (F(6,52)=3.0, p<0.05) and AUC (F(6,52)=2.8, p<0.05). Post-hoc MANOVA showed a significant decrease of the EMGₘₐₓ by morphine treatment at doses of 0.3, 1.5 and 3 mg/kg (p<0.05). Post-hoc MANOVA showed a significant decrease of the AUC by morphine treatment at doses of 1.5 and 3 mg/kg (p<0.05) and a tendency in AUC reduction by 0.3 mg/kg morphine treatment (p=0.07).

Passive Avoidance Test

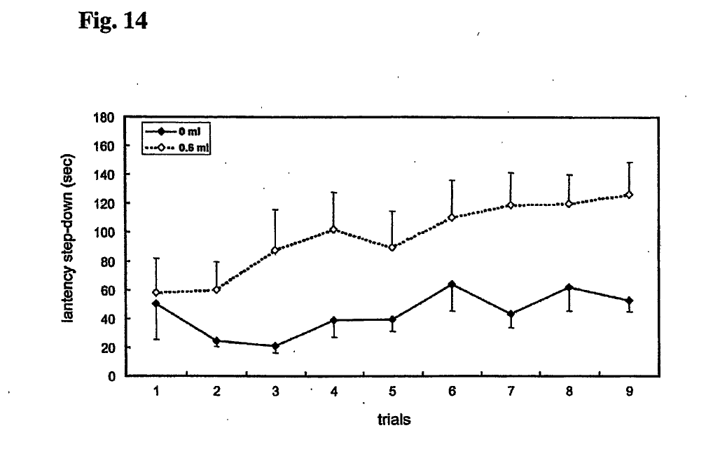

To evaluate the afferent pathway conveying acute duodenal nociception and cortical pain perception, one can stucy the animal behavioral responses to pain. In this study we employed a passive avoidance test to evaluate noxious related behaviour. In agreement with previous studies, our study shows that duodenal distension increases the step-down latency (FIG. 14 shows the step-down latency of rats in the passive avoidance test on day 2. Duodenal distention with a volume of 0.6 ml resulted in a significant increase in the step-down latency as compared to non-distended rats (F(1,10)=7.3, p<0.05)). Taken together with the above mentioned behavioral data, the present study indicates that duodenal distention in our animal model is perceived as a noxious, aversive stimulus.

### CONCLUSION

The present data show that radiotelemetry is an adequate tool to measure visceromotor and cardiovascular responses to visceral pain continuously and simultaneously in the conscious and freely moving rat, without additional handling-related or restraint stress. Duodenal distention induced “discomfort” and “pain” behavior and a volume-dependent visceromotor and cardiovascular response in the rat.

The staircase distention model is suitable for studying the threshold of aversive behavioral (grooming abdominal region, stretching), visceromotor (abdominal contractions, increase in EMG amplitude and AUC) and pseudoaffective (MAP and HR) responses to duodenal distention. The cardiovascular and visceromotor responses to duodenal distention are both useful measures of visceral nociception and abolition of both responses by a drug is predictive of antinociceptive efficacy. Both the staircase and phasic distention models are suitable for studying the potency of new pharmacological compounds on reversing visceral nociception.

1. An animal model for measuring visceral pain comprising a balloon catheter and an implantable sensor module having transcutaneous telemetring ability.

2. An animal model according to claim 1 wherein the balloon catheter is an implantable balloon catheter.

3. An animal model according to claim 2 wherein the implantable balloon cathether comprises fixation means.

4. An animal model according to claim 2 wherein the balloon catheter is implanted into the duodenum.

5. An animal model according to claim 1, wherein the implantable sensor module is capable of accepting a plurality of input signals.

6. An animal model according to claim 5 wherein the implantable sensor module is set up to receive both visceromotor and pseudoaffective responses of the test animal.

7. An animal model according to claim 5 wherein the implantable sensor comprises at least two input ports.

8. An animal model according to claim 5 wherein the implantable sensor is connected to a bipolar electrode pair and a blood catheter.

9. A balloon catheter comprising a biocompatible tubing (1) closed at one end with elastic material (2), characterized in that the elastic material is attached to the biocompatible tubing at a position (3) proximal from the tube end (4).

10. A balloon catheter according to claim 9 wherein the elastic material is also attached at the end of the biocompatible tubing (5) and said tubing end is rigidly sealed (6), further comprising a number of holes (7) distal from attachment point (3).

11. A balloon catheter according to claim 9 further comprising fixation means that are positioned proximal from the tube end.

12. A method of measuring visceral pain comprising:
implanting a balloon catheter according to claims 9;
implanting an implantable sensor module having transcutaneous telemetering ability;
monitoring telemetric signals from the implantable sensor with an external module; and
processing the telemetered signals.

13. A method according to claim 12 wherein the balloon catheter is implanted in the duodenum of the test animal; and wherein the implantable sensor module is set up to receive both visceromotor and pseudoaffective responses of the test animal.

14. A method according to claims 12 further comprising means for introducing a measured volume of inflation medium through the proximal end of the balloon catheter.

15. A method according to claim 14 wherein a syringe is used for introducing the measured volume of inflation medium.

16. A method according to claim 13 further comprising introducing a measured volume of inflation medium through the proximal end of the balloon catheter.

17. A method according to claim 16 wherein a syringe is used for introducing the measured volume of inflation medium.

18. An animal model according to claim 3 wherein the fixation means comprises two nodes to fixate the catheter.

19. A method of measuring visceral pain comprising:
implanting a balloon catheter according to claim 10;
implanting an implantable sensor module having transcutaneous telemetering ability;
monitoring telemetric signals from the implantable sensor with an external module; and
processing the telemetered signals.

20. A method of measuring visceral pain comprising:
implanting a balloon catheter according to claim 11;
implanting an implantable sensor module having transcutaneous telemetering ability;
monitoring telemetric signals from the implantable sensor with an external module; and
processing the telemetered signals.
